(19) United States
(12) Patent Application Publication
(10) Pub. No.: US 2014/0335585 A1
(43) Pub. Date: Nov. 13, 2014

(54) METHOD OF REUTILIZATION OF CARBON DIOXIDE FROM EMISSIONS

(71) Applicant: Alexander M. Chirkov, Lincoln, RI (US)

(72) Inventor: Alexander M. Chirkov, Lincoln, RI (US)

(73) Assignee: HILLWINDS ENERGY DEVELOPMENT CORPORATION, Fairfield, CT (US)

(21) Appl. No.: 14/237,971

(22) PCT Filed: Aug. 30, 2012
(86) PCT No.: PCT/US12/52957

§ 371 Date: Jun. 11, 2014

Related U.S. Application Data
(60) Provisional application No. 61/575,935, filed on Aug. 31, 2011.

(51) Int. Cl. C12P 5/02

(52) U.S. Cl. CPC C12P 5/023
USPC 435/167; 435/294.1

(57) ABSTRACT

This invention relates to apparatuses and methods for the reduction of carbon dioxide emissions resulting from combustion using a bioreactor that includes methanogenic bacteria or genetically modified algae.

### FIELD OF THE INVENTION

The invention relates to reduction of carbon dioxide emissions resulting from combustion, and more particularly reduction of carbon dioxide emissions resulting from combustion using a bioreactor that includes methanogenic bacteria or genetically modified algae.

### BACKGROUND OF THE INVENTION

Air pollution and global warming are two major environmental challenges of the developed and developing world. Carbon dioxide produced as a result of combustion is recognizable and known factor in the emission of “greenhouse” gases. Carbon dioxide is an odorless colorless gas that also results from the respiration of biological systems that are a normal part of earth's ecological system. However, excess production of carbon dioxide as a result of industrial development is considered one of the major factors in global warming and ocean acidification, both of which pose major environmental challenges to the earth.

The decrease of carbon dioxide production is part of a complex social, economical, scientific and technological objective. The existing ecological system formed on the earth millions of years ago by eliminating excesses of carbon production using photosynthesis and dissolving carbon dioxide in water. Industrial developments by humans, including advances such as power plants, automobile transportation, and cement production, have increased the carbon dioxide level by a thousand times to the point where the ecological system can no longer balance itself Accordingly, carbon sequestration (e.g., the process of gathering carbon from the atmosphere or other sources) has become an emerging technology that could affect the healthy environment necessary for the growth of the society. There have been several approaches developed to sequester or reduce the output of carbon:

A. Geological carbon dioxide sequestration—Geological carbon dioxide sequestration utilizes technology that stores a significant quantity of carbon dioxide in geological cavities by injecting the gas underground. While the advantages are self-evident, there are also a few disadvantages to this approach, such as high cost and uncertain results.

B. “Green Technologies”—Utilizing energy technologies that emit fewer pollutants are broadly categorized as “Green Technology”. For example, clean burning technology that decreases the production of carbon dioxide per unit of power or product. This also includes socio-behavioral modifications that decrease carbon dioxide production and increase its natural utilization such as forest preservation, limitations on car usage, solar panels, wind mills and etc.

C. Biological sequestration—This methodology is based on increasing carbon dioxide utilization by managing the conditions of photosynthesis and increasing energy derived from recirculated products. This methodology includes, for example, mass production of algae to be used for its constituent components such as fat, hydrocarbons, and proteins.

D. Chemical sequestration—This technology sequesters carbon dioxide in a non-soluble, harmless solution such as calcium bicarbonate. The major disadvantages of this method include high energy consumption, high cost, and the unavailability of reactive calcium.

A review of the natural biological pathway of carbon dioxide production and utilization shows its correlation and relationships between its source and methane production pathways. Methane and carbon dioxide are two major components of biogas which is generated as a result of the biological break down of organic material most commonly known as anaerobic digestion. Methane can combust or oxidize in order to release energy, and therefore can be a source of fuel.

It is known that methane can be produced from carbon dioxide by direct chemical reaction. There are two major chemical approaches to producing methane:

1. The Sabatier Reaction (name after the French chemist) involves the reaction of hydrogen with carbon dioxide under high temperature and pressure while in the presence of a nickel or an alumina oxide catalyst. This method is used to regenerate water on space stations. However, it is extremely expensive.

2. The Fisher-Tropsch process is used mostly to produce liquid hydrocarbons from a mixture of carbon monoxide and hydrogen. Methane can be produced in this process as an intermediate step in several reactions.

In addition to chemical reactions, methane can be produced by biological systems (bacteria) under both anaerobic and aerobic conditions. Bacterial methane is formed from the degradation of long chain hydrocarbons and carbon dioxide. Methanogens are microorganisms that produce methane (for example, Methanopyrus kandleri and Methanosacina barkeri). Superoxide dismutase enzyme present in different species of bacteria allows methane to be produced in aerobic conditions. There is data that shows the possibility of producing methane from vegetation. The mechanisms in this process are not indentified, but there is data that verifies an excess of methane accumulation in areas of rice production. Methanogenic bacteria, which use carbon dioxide as a source of carbon and hydrogen as a reducing agent, use the enzyme in the cytoplasm as well as enzymes within the bacterial wall that produce an electrochemical gradient across a membrane. In theory, this could be used in scaled-up commercial processes. However, bacteria have several disadvantages in industrial methane processes. First, bacteria generally have a slow growth rate, a short lifespan, and require significant solid substances for growth. On the other hand, other organisms such as algae do not have these disadvantages. For example, algae can be cultivated to a higher density, thus resulting in higher productivity for any given volume (see, for example, FIG. 1 which shows increased algal biomass production as compared to bacteria). In addition, many algae species have genomes that are fully sequenced and characterized, and that are small so that they lend themselves to genetic manipulation (e.g., Prochlorococcus sp., approx. 1.7 Mb, Noctroc punctiforme, .approx. 9.1 Mb). It would be advantageous if selected, genetically modified algae could be used to replace certain species of bacteria in industrial processes, such as methane production and carbon dioxide utilization.

### SUMMARY OF THE INVENTION

In one aspect, the present invention is directed to an apparatus for reducing carbon dioxide emissions produced during combustion, comprising: (a) a combustion chamber, the combustion chamber; (b) a nitrogen removal system in fluid communication with the combustion chamber; (c) a gas cooling system in fluid communication with the nitrogen removal system; and (d) a bioreactor in fluid communication with the gas cooling system, the bioreactor comprising one or more active plates, the active plates each comprising methanogenic bacteria or genetically modified algae positioned on a semipermeable membrane, wherein the methanogenic bacteria are selected from the group consisting of Methanopyrus kandleri, Methanosarcina barkeri, and combinations thereof; and the genetically modified algae are selected from the group consisting of genetically modified Cyanophyta.

In another aspect, the present invention is directed to a method for converting carbon dioxide to methane, comprising the steps of combusting fuel in a combustion chamber to produce exhaust gas, the exhaust gas comprising carbon dioxide; and (b) transferring the carbon dioxide to a bioreactor to convert the carbon dioxide to methane, the bioreactor comprising one or more active plates, the active plates each comprising methanogenic bacteria or genetically modified algae positioned on a semipermeable membrane, wherein the methanogenic bacteria are selected from the group consisting of Methanopyrus kandleri, Methanosarcina barkeri, and combinations thereof; and the genetically modified algae are selected from the group consisting of genetically modified Cyanophyta.

### BRIEF DESCRIPTION OF THE DRAWINGS

The invention will be better understood from the following detailed description and examples, taken in conjunction with the drawings, in which:

FIG. 1 is a graph showing increased algal biomass production as compared to bacteria;

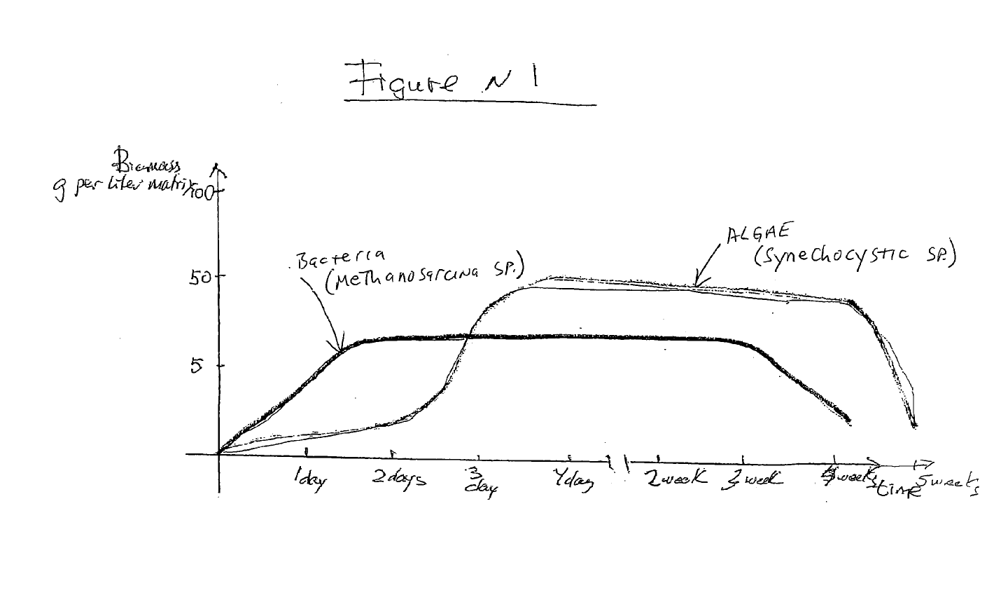

FIG. 2 is schematic view of an active plate of the bioreactor of the invention;

FIG. 3 is a schematic diagram of the method of reutilization of carbon dioxide according to the invention; and

FIG. 4 is a schematic diagram of several plates of the bioreactor of the invention.

### DETAILED DESCRIPTION OF THE INVENTION

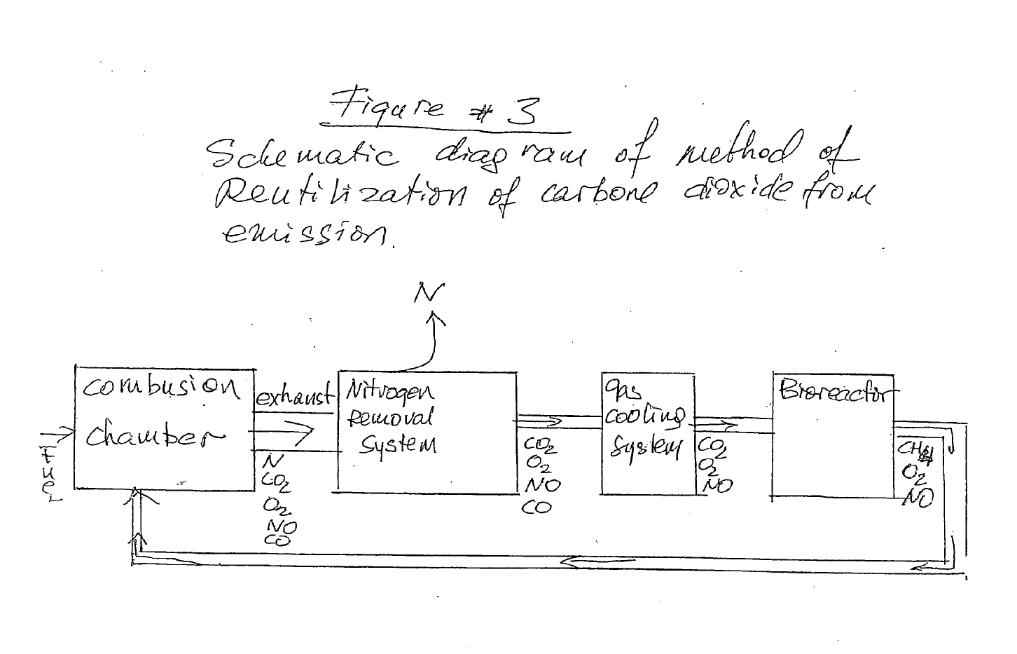

The method and apparatus of the invention reduces carbon dioxide atmospheric emissions produced during combustion by converting them into methane using a biological reactor and burning the resulting methane in a cyclical closed system. FIG. 3 shows a general schematic outline of the components used in the method of the invention. Briefly, the invention includes a combustion chamber, a nitrogen removal system, a gas cooling system, and a bioreactor. As shown in FIG. 3, fuel is added to the combustion chamber, which produces exhaust gases (nitrogen, carbon dioxide, oxygen, nitric oxide, carbon monoxide, etc.). Any fuel known in the combustion arts can be used as fuel in this invention, including but not limited to natural gas, coal, hydrocarbons (oil), and the like. Carbon dioxide constitutes about 8-15% of the exhaust gases produced by the combustion, while nitrogen, oxygen, nitric oxide, carbon monoxide, and other gases constitute about 80% of the exhaust. A majority of the nitrogen-containing gases are preferably removed by the nitrogen removal system prior to introduction into the bioreactor.

After the separation and cooling of the gases to approximately 70-80° F. and approximately 50 psi, the carbon dioxide is transferred through a series of closed loop bioreactors that convert carbon dioxide to methane and oxygen. The active component in the bioreactor is a methanogenic bacteria or a genetically modified algae (cyanobacteria). Methanogenic bacteria useful in the method of the invention include hydrogenotrophic bacteria such as Methanopyrus kandleri, Methanosarcina barkeri, and the like, which can be found in open environment (e.g., mostly in anaerobic conditions in wetland, marine sediment, and in rock). It is possible to grow such bacteria in the laboratory on known anaerobic media, such as the GasPak System. The cyanobacteria (also known as blue-green algae) useful in the method of the invention include Cyanophyta (for example, unicellular form Chroococcales sp. or Synechocystic sp.), and can be found in soil, or fresh or salt water. These species are generally easy to grow in photo bioreactors with prolonged life span compared with bacteria. The cyanobacteria used in the method of the invention are genetically modified to include genes that produce bacterial proteins that convert carbon dioxide to methane.

The bacterial protein responsible for conversion of carbon dioxide to methane has been identified in a hydrogenotrophic population of Methanopyrus kandleri. The protein is cytoplasmic and has an approximate MW of 200,000. Using conventional techniques, the gene that codes for this protein can be inserted into the cyanobacteria to produce an algal strain that includes the capability to convert carbon dioxide to methane. For example, in one embodiment, the isolated gene can be amplified using PCR techniques and inserted into the target genome using known gene splicing technology. After modification to include gene elements that provide better expression and effectiveness, the gene is inserted into a bacterial plasmid using conventional gene insertion techniques, and the resulting plasmid is introduced into the algal genome, again using known selection protocols. Once transformed algae are identified, they are grown to level of biomass that is useful in the bioreactor.

In one embodiment, gene determination was made by methods of exclusion based on population study. The data showed that a colony of Methanopyrus Kandleri without selected fragment of DNA was unable to support constant methanogenesis on any level under constant carbon dioxide replacement. Gene sequestration was not performed. Gene optimization including study of critical factors involving different stages of protein expression, such as codon adaptivity, mRNA structure and various cis-elements in transcription and translation was not performed. A study of the transformed cyanobacteria showed that the integrated genome was capable to transfer carbon dioxide in methane at the level of 3% without decreasing of life span of algae population.

The effectiveness of enzyme protein complexes extracted from cytoplasm of Methanopyrus Kandleri was determined through direct measurement of transformation of carbon dioxide to methane in acid media (pH 5.4), temperature of 32° C. under constant flow of carbon dioxide under 50 psi. (3.5 atm). In one exemplary embodiment, the rate of conversion was 4% under 20 minute cycles in excess amount of phosphocreatine.

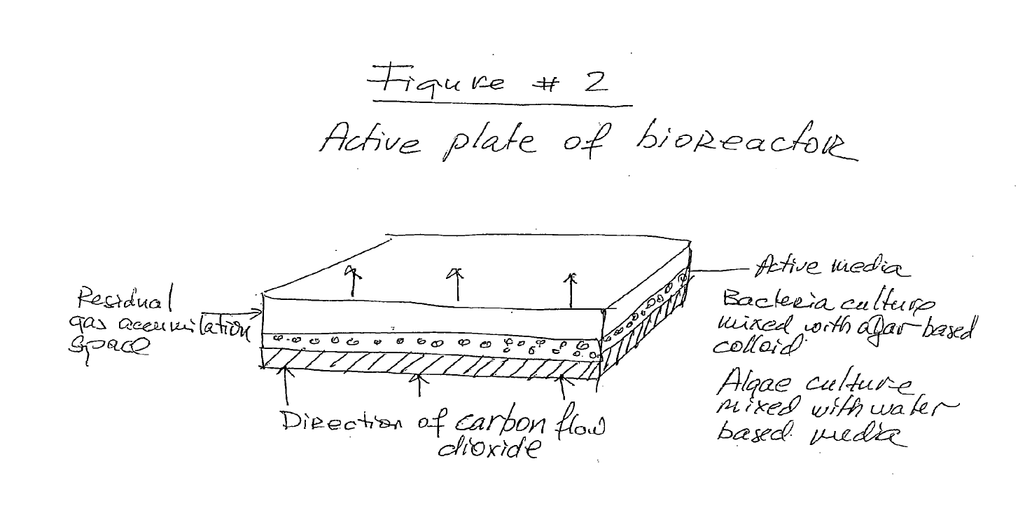

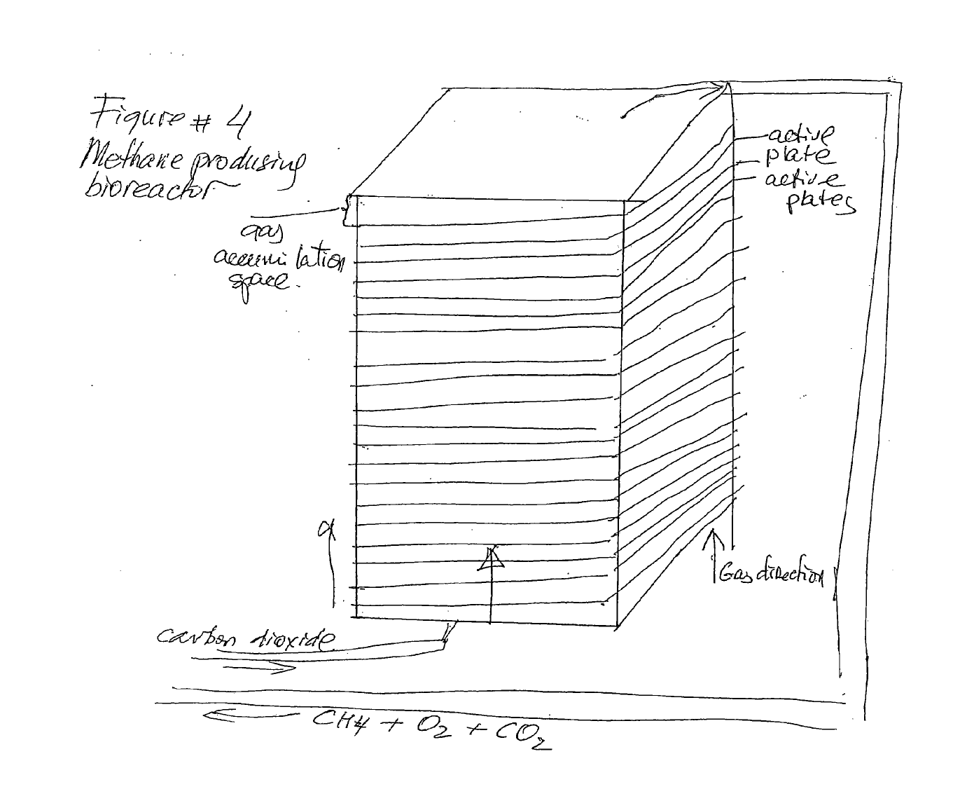

Referring again to the method of the invention, the active component (e.g., methanogenic bacteria or genetically modified algae) is placed on a semipermeable membrane with one directional gas flow, from the bottom tray up (FIG. 2). The closed loop bioreactor consists of a plurality of these vertically oriented multiple platforms (trays) which contain the methanogenic organisms (bacteria or genetically modified algae) on a firm gas permeable base (FIG. 4). During use, the carbon dioxide is circulated through the base of this platform and the active media, starting from the bottom and moving toward the top, where it is transformed to methane. The quantity of bioreactors and their active components is determined by the volume of the primary carbon dioxide availability. The resulting mixture of methane, oxygen and residual carbon dioxide is redirected into a combustion chamber.

Although not wishing to be bound by any particular material or size, the plate shown in FIG. 2 may be made of any material that is suitable for use in a bioreactor. Preferably, stainless steel is used since it is resistant to the corrosive effects of carbon dioxide. In one embodiment, the plate is 2 inches deep with windows at the base, and has one preferred size of about 3 foot wide and 5 foot long. The window at the base is 12 inches by 12 inches. The window is covered by a semipermeable membrane(e.g., cellulose and the like) to allow gases to move through the membrane. Each plate is generally constructed with a first layer being the semipermeable membrane, a second layer that contains nutrient media agar for bacteria and/or water-based algae, and a third layer of open space for gas accumulation.

While the amount of organisms may be any effective amount, preferably, the plates contain approximately 20 g of bacteria (dry mass) per liter of media, and approximately 40-60 g algae (dry mass) per liter of media. The nutrient media layer preferably contains all the nutrients needed by the organisms for sustained growth and functioning in a culture or biomass for at least for 4 weeks, and such nutrients are well known in the art. The algae may be optionally supplemented with artificial light for growth, depending on the species, preferably in the range of 400-600 nm. In terms of operation, the rate of the transformation of carbon dioxide to methane using the bioreactor is preferably approximately 2-4% of gas passing through the tray with flow rate of 5 liter per minute.

Thus the proposed method of carbon dioxide utilization is a combination of a natural system component with an industrial component. The bacterial or algae biomass is an active agent that produces methane in the natural system component. The generated methane is used in the industrial component as fuel and burned in the same combustion chamber that produces carbon dioxide, thus decreasing the consumption of primary fuel. Since the biomass is used continuously in a closed cyclical system, this method eliminates the costly procedure of processing the biomass for future usage. Therefore, the energy efficiency of the biomass significantly increases while simultaneously decreasing the expense. Overall, the method can significantly decrease the atmospheric release of carbon dioxide in an energy-generating system by sequestering carbon and utilizing it to produce methane as fuel in the production cycle.

What is claimed is:

1. An apparatus for reducing carbon dioxide emissions produced during combustion, comprising:
(a) a combustion chamber,
(b) a nitrogen removal system in fluid communication with said combustion chamber;
(c) a gas cooling system in fluid communication with said nitrogen removal system; and
(d) a bioreactor in fluid communication with said gas cooling system, said bioreactor comprising one or more active plates, said active plates each comprising methanogenic bacteria or genetically modified algae positioned on a semipermeable membrane, wherein
said methanogenic bacteria are selected from the group consisting of Methanopyrus kandleri, Methanosarcina barkeri, and combinations thereof; and
said genetically modified algae are selected from the group consisting of genetically modified Cyanophyta.

2. The apparatus of claim 1, wherein said genetically modified Cyanophya are selected from the group consisting of Chroococcales species, Synechocystic species, and combinations thereof.

3. The apparatus of claim 1, wherein said genetically modified algae are genetically modified to convert carbon dioxide to methane.

4. The apparatus of claim 1, wherein said bioreactor contains 20 grams of bacteria (dry mass) per liter of media.

5. The apparatus of claim 1, wherein said bioreactor contains 40-60 grams algae (dry mass) per liter of media.

6. The apparatus of claim 1, wherein said semipermeable membrane is cellulose.

7. The apparatus of claim 1, wherein said one or more active plates further comprise a nutrient media.

8. A method for converting carbon dioxide to methane, comprising the steps of:
(a) combusting fuel in a combustion chamber to produce exhaust gas, said exhaust gas comprising carbon dioxide;
(b) transferring said carbon dioxide to a bioreactor to convert said carbon dioxide to methane, said bioreactor comprising one or more active plates, said active plates each comprising methanogenic bacteria or genetically modified algae positioned on a semipermeable membrane, wherein
said methanogenic bacteria are selected from the group consisting of Methanopyrus kandleri, Methanosarcina barkeri, and combinations thereof; and
said genetically modified algae are selected from the group consisting of genetically modified Cyanophyta.

9. The method of claim 8, wherein said genetically modified Cyanophya are selected from the group consisting of Chroococcales species, Synechocystic species, and combinations thereof.

10. The method of claim 8, wherein said genetically modified algae are genetically modified to convert carbon dioxide to methane.

11. The method of claim 8, wherein said bioreactor contains 20 grams of bacteria (dry mass) per liter of media.

12. The method of claim 8, wherein said bioreactor contains 40-60 grams algae (dry mass) per liter of media.

13. The method of claim 8, wherein said semipermeable membrane is cellulose.

14. The method of claim 8, wherein said one or more active plates further comprise a nutrient media.

15. The method of claim 8, wherein said bioreactor converts approximately 2-4% of carbon dioxide to methane.

16. The method of claim 8, further comprising the step of transferring said methane to said combustion chamber.
